(19) United States
(12) Patent Application Publication
(10) Pub. No.: US 2012/0214682 A1
(43) Pub. Date: Aug. 23, 2012

(54) MICRORNA SIGNATURES INDICATIVE OF IMMUNOMODULATING THERAPY FOR MULTIPLE SCLEROSIS

(76) Inventors: Douglas W. Bigwood, Madison, CT (US); Eric M. Eastman, Highland, MD (US)

(21) Appl. No.: 13/359,675

(22) Filed: Jan. 27, 2012

Related U.S. Application Data
(60) Provisional application No. 61/437,382, filed on Jan. 28, 2011.

(51) Int. Cl. C40B 30/04; C12Q 1/68

(52) U.S. Cl. 506/9; 435/6.11

(57) ABSTRACT

The present invention provides methods, systems, and kits for evaluating multiple sclerosis (MS) in a patient. Particularly, the invention provides convenient miRNA-based tests for evaluating a patient for MS, including for diagnosing MS, for excluding MS as a diagnosis, and for monitoring the course of disease or efficacy of treatment, including evaluation of immunomodulating therapy.

### CROSS-REFERENCE TO RELATED APPLICATIONS

This application claims priority to and the benefit of U.S. Provisional Application No. 61/437,382, filed Jan. 28, 2011, the disclosure of which is hereby incorporated by reference in its entirety.

### FIELD OF THE INVENTION

The present invention relates to evaluating multiple sclerosis (MS) and/or treatment for MS using miRNA profiles, to thereby assist in the diagnosis, prognosis, and/or monitoring of treatment for MS.

### BACKGROUND OF THE INVENTION

Multiple sclerosis (MS) is a disease that affects the central nervous system, and can range from relatively benign to somewhat disabling to devastating. In MS, the myelin surrounding nerve cells is damaged or destroyed, impacting the ability of the nerves to conduct electrical impulses to and from the brain, and leaving scar tissue called sclerosis. These damaged areas are also known as “plaques” or “lesions.”

The first symptoms of MS typically appear between the ages of 20 and 40, and include blurred or double vision, red-green color distortion, or even blindness in one eye. Most MS patients experience muscle weakness in their extremities and difficulty with coordination and balance. In severe cases, MS can produce partial or complete paralysis. Paresthesias (numbness, prickling, or “pins and needles”), speech impediments, tremors, and dizziness are frequent symptoms of MS. Approximately half of MS patients experience cognitive impairments.

Diagnosing MS is complicated, because there is no single test that can confirm the presence of MS. The process of diagnosing MS typically involves criteria from the patient's history, a clinical examination, and one or more laboratory tests, with all three often being necessary to rule out other possible causes for symptoms and/or to gather facts sufficient for a diagnosis of MS.

Magnetic resonance imaging (MRI) is a preferred test. An MRI can detect plaques or scarring possibly caused by MS. However, an abnormal MRI does not necessarily indicate MS, as lesions in the brain may be associated with other disorders. Further, spots may also be found in healthy individuals, particularly in healthy older persons. These spots are called UBOs, for unidentified bright objects, and are not related to an ongoing disease process. In addition, a normal MRI does not absolutely rule out the presence MS. About 5% of individuals who are confirmed to have MS on the basis of other criteria, have no brain lesions detectable by MRI. These individuals may have lesions in the spinal cord or may have lesions that cannot be detected by MRI.

While a diagnosis of MS might be based on an evaluation of symptoms, signs, and the results of an MRI, additional tests may also be ordered. These include tests of evoked potential, cerebrospinal fluid, and blood. For example, cerebrospinal fluid is sampled by a lumbar puncture, and is tested for levels of immune system proteins and for the presence of an antibody staining pattern called “oligoclonal bands.” Oligoclonal bands indicate an immune response within the central nervous system and are found in the spinal fluid of 90-95% of individuals with MS. However, oligoclonal bands are also associated with diseases other than MS, and therefore the presence of oligoclonal bands alone is not definitive of MS.

There is likewise no definitive blood test for MS, but blood tests can exclude other possible causes for various neurologic symptoms, such as Lyme disease, collagen-vascular diseases, rare hereditary disorders, and AIDS.

Diagnosing MS generally requires: (1) objective evidence of at least two areas of myelin loss, or demyelinating lesions, “separated in time and space” (lesions occurring in different places within the brain, spinal cord, or optic nerve-at different points in time); and (2) all other diseases that can cause similar neurologic symptoms have been objectively excluded. Until (1) and (2) are satisfied, a physician does not make a definite diagnosis of MS.

Depending on the clinical problems present when an individual sees a physician, one or more of the tests described above might be performed. Sometimes tests are performed several times over a period of months to help gather the necessary information. A definite MS diagnosis must satisfy the McDonald criteria, named for the distinguished neurologist W. Ian McDonald who sparked society-supported efforts to make the diagnostic process for MS faster and more precise.

There are a few distinct clinical courses for MS, referred to as relapsing-remitting MS, secondary-progressive MS, progressive-relapsing MS, and primary progressive MS. Relapsing-remitting MS is characterized by clearly-defined, acute attacks (relapses), usually with full or partial recovery, and no disease progression between attacks. Secondary-progressive MS is initially relapsing-remitting but then becomes continuously progressive at a variable rate, with or without occasional relapses along the way. The disease-modifying medications are thought to provide benefit for those who continue to have relapses. Primary progressive MS may be characterized by disease progression from the beginning with few or no periods of remission. Progressive-relapsing MS is characterized by disease progression from the beginning, but with clear, acute relapses along the way.

There are several options available for treating individuals diagnosed with MS. Beta-interferon (Avonex, Betaseron, Rebif) has been approved to treat MS. Interferons are also made by the body, mainly to combat viral infections. Interferons have been shown to decrease the worsening or relapse of MS, however disease progression remains unaffected and the side effects of interferons are poorly tolerated. Glatiramer acetate (Copaxone) is a mixture of amino acids that has been shown to decrease the relapse rates of MS by 30%, and appears to also have a positive effect on the overall level of disability. Glatiramer acetate is better tolerated than the interferons and has fewer side effects. Glatiramer acts by binding to major histocompatibility complex class II molecules and competing with MBP and other myelin proteins for such binding and presentation to T cells. Natalizumab (Tysabri) is a monoclonal antibody that binds to alpha-4-integrin on white blood cells and interferes with their movement from the bloodstream into the brain and spinal cord.

An object of the present invention is to provide a convenient diagnostic test for a more objective, definitive, and rapid diagnosis of MS. Another object of the invention is to provide a diagnostic test for monitoring MS progression, adequacy of treatment, and/or response to treatment, including immunomodulating treatment such as interferon therapy (e.g., Avonex).

Other objects of the invention will be apparent from the following description of the invention.

### SUMMARY OF THE INVENTION

The present invention provides methods, systems, and kits for evaluating a demyelinating disease, such as multiple sclerosis (MS) in a patient. Particularly, the invention provides convenient miRNA-based tests for evaluating a patient for MS, including for diagnosing MS, for excluding MS as a diagnosis, and for monitoring the course of disease or efficacy of treatment.

In one aspect, the invention provides a method for evaluating a demyelinating disease in a patient. For example, the patient may be suspected of having MS, either due to the presence of demyelinating lesions consistent with MS, or the presence of symptoms of a neurologic and/or immunologic disorder consistent with MS. The patient may be undergoing treatment for the demyelinating disease (e.g. MS), such as immunomodulating therapy. In some embodiments, the patient is receiving interferon therapy (e.g., Avonex). In this aspect, the method comprises preparing a miRNA profile from a biofluid sample of the patient (e.g., for samples taken before and/or after initiating treatment), and determining the presence or absence of a miRNA signature indicative of a response to treatment with the immunomodulating agent (e.g., in samples taken before and/or after treatment.) The miRNA profile comprises the level or abundance of at least 2 miRNAs of Table 1, Table 2, and/or Table 3.

The sample, which may be obtained pre- and/or post- treatment for MS, is a biofluid sample, such as a serum or plasma sample (e.g., a cell-free blood sample), or in other embodiments, a whole blood or peripheral blood mononuclear cell (PBMC) sample. In still other embodiments, the sample is urine, saliva, or cerebrospinal fluid. In certain embodiments, the sample is a serum sample, which may be collected with the use of a serum separator tube, “red-top” tube or clot activator tube. RNA may be subsequently isolated from the serum for miRNA profiling. The miRNA profile is determined by an amplication and/or hybridization-based assay, including, for example, Real-Time PCR (e.g., TaqMan). Other exemplary detection platforms, including direct miRNA capture and miRNA hybridization arrays, are described herein.

The miRNA profile represents the absolute or relative level or abundance of miRNAs present in the sample, and comprises levels for a plurality of miRNAs of Table 1, Table 2, and/or Table 3. In various embodiments, the miRNA profile comprises the level of at least 4, 6, 8, 10, 20, 50, 75, or more miRNAs of Table 1, Table 2, and/or Table 3. In certain embodiments, the miRNA profile is prepared with the use of a custom kit or array, e.g., to allow particularly for the profiling of miRNAs associated with MS. Such profiling may involve determining the level of 150 miRNAs or less, or in other embodiments 100 miRNAs or less, 75 miRNAs or less, 50 miRNAs or less, 25 miRNAs or less, or 10 miRNAs or less, and including miRNAs of Table 1, Table 2, and/or Table 3.

The miRNA profile is evaluated for the presence or absence of a miRNA signature indicative of a response to treatment (e.g., for MS). The presence or absence of the signature may be determined by any suitable algorithm, which in some embodiments involves determining whether the miRNA levels are above or below threshold levels that are indicative of MS, or indicative of treatment or response to treatment for MS. The signature may be indicative of a positive response to interferon therapy. In some embodiments, the threshold miRNA levels are set to include about the top or bottom 10% of expression levels as determined in a suitable population of MS patients and healthy controls. Alternatively, the algorithm may involve classifying a sample based upon Mean and/or Median miRNA levels in MS patients being treated for MS versus a non-MS population (e.g., a population of healthy controls or population of patients with diseases other than MS) or a popution that is treatment naive.

The invention thereby provides a predictor for determining a response to immunomodulatory treatment for a demyelinating disease such as MS, including the efficacy of treatment for interferon therapy (e.g. Avonex).

In another aspect, the invention provides a method for preparing a miRNA profile indicative of the presence or absence of multiple sclerosis (MS), such as relapsing remitting MS, or indicative of a positive response to interferon therapy. The method comprises preparing a miRNA profile from a biofluid, such as a serum or plasma sample (e.g., a cell-free blood sample), of a patient suspected of having MS. The sample may be taken before and/or after treatment, or periodically during treatment to monitor treatment efficacy. The miRNA profile comprises the level of 150 miRNAs or less, and includes at least 2 miRNAs of Table 1, Table 2, and/or Table 3. In certain embodiments, the miRNA profile comprises the level of at least 4, or at least 6, or at least 8, or at least 10, or at least 20 miRNAs of Table 1, Table 2, and/or Table 3. The miRNA profile may be prepared with the use of a custom kit or array, e.g., to allow particularly for the profiling of miRNAs associated with MS. Such profiling may involve determining the level of 100 miRNAs or less, 75 miRNAs or less, 50 miRNAs or less, 25 miRNAs or less, or 10 miRNAs or less, including miRNAs of Table 1, Table 2, and/or Table 3.

The miRNA profile may be determined by a variety of detection platforms as described herein, including Real-Time PCR (e.g., TaqMan).

In another aspect, the invention provides a kit or test for preparing the miRNA profiles. The kit or test may be configured for a variety of miRNA detection platforms as described herein.

Other aspects and embodiments of the invention will be apparent to the skilled artisan in view of the following detailed description.

### DETAILED DESCRIPTION OF THE INVENTION

The present invention provides methods, systems, and kits for evaluating a demyelinating disease, such as multiple sclerosis (MS). The invention provides convenient miRNA-based tests for evaluating MS in patients, and for monitoring treatment. Such patients may be known to have MS, may be suspected of having MS on the basis of one or more MS-like symptoms or results from one or more MS-related clinical exams, or may be beginning or undergoing treatment for MS (e.g., with interferon therapy). In the various aspects of the invention monitors the progression of disease, or determines efficacy of interferon treatment.

MicroRNAs (miRNAs) are small (22 nt on average) non-coding RNA molecules that have been identified in plants, animals, and other organisms. miRNAs are involved in the post-transcriptional regulation (e.g., silencing) of gene expression, and act by binding to complementary sequences in target messenger RNA transcripts (mRNAs). The human genome may encode over 1000 different miRNAs. miRNAs are associated with fundamental biological processes, including hematopoietic differentiation, cell cycle regulation, metabolism, cardiovascular biology, and immune function. miRNAs can also be associated with the presence and/or progression of disease. See, Calin et al., A microRNA signature associated with prognosis and progression in chronic lymphocytic leukemia, N. Engl. J. Med. 353:1793-1801 (2005); Barbarotto et al., MicroRNAs and cancer: profile, profile, profile. Int. J. Cancer 122:969-977 (2008). The present invention is based, in-part, on the association of miRNA levels with MS.

### Methods For Diagnosing MS

Generally, the patient is suspected of having MS. For example, the patient may be suspected of having MS on the basis of neurologic and/or immunologic symptoms consistent with MS, e.g., after an initial physician's exam. The patient may, in some embodiments, be positive for the presence of oligoclonal bands. In these or other embodiments, the patient may have CNS lesions characteristic of MS, which are observable on an MRI. In certain embodiments, the patient has not undergone treatment for MS, but in some embodiments, the patient is already undergoing treatment, such as treatment with Beta-interferon, Glatiramer acetate, and Natalizumab.

Thus, the patient may have one or more presumptive signs of a multiple sclerosis. Presumptive signs of multiple sclerosis include for example, altered sensory, motor, visual or proprioceptive system with at least one of numbness or weakness in one or more limbs, often occurring on one side of the body at a time or the lower half of the body, partial or complete loss of vision, frequently in one eye at a time and often with pain during eye movement, double vision or blurring of vision, tingling or pain in numb areas of the body, electric-shock sensations that occur with certain head movements, tremor, lack of coordination or unsteady gait, fatigue, dizziness, muscle stiffness or spasticity, slurred speech, paralysis, problems with bladder, bowel or sexual function, and mental changes such as forgetfulness or difficulties with concentration, relative to medical standards.

The sample, which may be obtained pre- and/or post- treatment for MS, is a biofluid sample, such as a cell-free blood sample (e.g., serum, plasma, or fraction thereof), or in other embodiments, is a whole blood sample or PBMC sample. In still other embodiments, the sample is urine, saliva, or cerebrospinal fluid collected from the patient. miRNAs have been detected, not only in association with blood cells, including PBMCs and platelets, but also in biofluid samples including serum, plasma, urine, and saliva. Hunter et al., Detection of microRNA Expression in Human Peripheral Blood Microvesicles, PloS One Vol. 3, Issue 11 (November 2008); Mitchell et al., Circulating microRNAs as stable blood-based markers for cancer detection, PNAS 105(30):10513-10518 (2008); and Hanke et al., A robust methodology to study urine microRNA as tumor marker: microRNA-126 and microRNA-182 are related to urinary bladder cancer, UrolOnc (Apr. 17, 2009). Thus, in some embodiments, the sample is a serum sample, and which is conveniently and reproducibly collected using, e.g., a serum separator tube or comparable device (e.g., red-top tube or clot activator tube). Various products for serum or plasma collection are well known and commercially available. For example, the serum separator tube may provide a draw volume of from about 2 to about 15 mL.

In some embodiments, RNA is extracted from the sample prior to miRNA processing for detection. RNA may be purified using a variety of standard procedures as described, for example, in RNA Methodologies, A laboratory guide for isolation and characterization, 2nd edition, 1998, Robert E. Farrell, Jr., Ed., Academic Press. In addition, there are various processes as well as products commercially available for isolation of small molecular weight RNAs, including mirVANA™ Paris miRNA Isolation Kit (Ambion), miRNeasy™ kits (Qiagen), MagMAX™ kits (Life Technologies), and Pure Link™ kits (Life Technologies). For example, small molecular weight RNAs may be isolated by organic extraction followed by purification on a glass fiber filter. Alternative methods for isolating miRNAs include hybridization to magnetic beads.

Alternatively, miRNA processing for detection (e.g., cDNA synthesis) may be conducted in the biofluid sample, that is, without an RNA extraction step.

The miRNA profile (and/or miRNA signature) is generated from samples using any of various techniques known in the art for quantifying miRNA levels, and exemplary detection platforms are described elsewhere herein. Briefly, such methods include, without limitation, polymerase-based assays, such as quantitative RNA-PCR, incuding real-time PCR (e.g., Taqman™), microarray or bead-based hybridization platforms, flap-endonuclease-based assays (e.g., Invader™), as well as direct miRNA capture. For example, miRNA expression can be quantified in a two-step polymerase chain reaction (PCR) process including reverse transcriptase PCR, followed by quantitative real-time PCR. For larger profiles, miRNAs can be hybridized to microarrays, beads, slides or chips. Various commercial products are available for quantifying miRNA levels including the TaqMan Low Density microRNA Array card (TLDA card) (Applied Biosystems Inc.).

The miRNA profile in this aspect of the invention comprises the absolute or relative level (or abundance) of miRNAs present in the sample, and includes the levels for a plurality of miRNAs of Table 1, Table 2, and/or Table 3. The nucleotide sequences of the miRNAs listed in Table 1, 2, and 3 are known, and these sequences are hereby incorporated by reference. In various embodiments, the miRNA profile comprises the level of at least about 4, 6, 8, 10, 20, 50, 75, or more miRNAs of Table 1, Table 2, and/or Table 3. miRNA levels may be expressed in accordance with the selected detection assay. For example, where Real-Time PCR (RT-PCR) is conducted, miRNA levels may be expressed in terms of cycle threshold (CT) values. CT values may be normalized as described herein. Alternatively, the profile may be determined by microarray analysis, and the miRNA levels expressed by relative hybridization signal intensity, as normalized for variables such as background, sample processing, and hybridization efficiency.

The miRNA profile may be prepared with the use of a custom kit or array, e.g., to allow particularly for the profiling of miRNAs associated with MS. Such profiling may involve determining the level of 150 miRNAs or less, or in other embodiments 100 miRNAs or less, 75 miRNAs or less, 50 miRNAs or less, 25 miRNAs or less, including 4, 6, 8, 10, 20, 50, 75, or more miRNAs of Table 1, Table 2, and/or Table 3. In some embodiments, at least 25%, or at least 50%, or at least 75% of the miRNAs of the profile are listed in Table 1, Table 2, and/or Table 3. In certain embodiments, the miRNA profile includes the level of miRNAs associated with a non-MS autoimmune disorder, inflammatory disorder, or infectious disease to better discriminate disease states having overlapping symptoms, such as systemic lupus erythematosus, Sjögren's syndrome, vasculitis, sarcoidosis, Behçet's disease, Lyme disease, syphilis, progressive multifocal leukoencephalopathy, herpes zoster, lysosomal disorder, adrenoleukodystrophy, and CNS lymphoma.

The method may further comprise determining the presence of at least one control RNA to normalize expression levels across samples. For example, the normalization control may be one or more exogenously added RNA(s) or miRNA(s) that are not naturally present in the sample. The normalization control in certain embodiments comprises an Arabidopsis miRNA, such as ath-miR-159a, and/or one or more human miRNAs not expressed in the sample undergoing analysis (e.g., serum). Alternatively or in addition, other methods of normalizing expression levels may be employed, such as normalizing based upon the Mean or Median level of all miRNAs on a given assay run. Methods for normalizing miRNA expression levels are described in Benes and Castoldi, Expression profiling of microRNA using real-time quantitative PCR, how to use it and what is available, Methods 50:244-249 (January 2010); Mestdagh et al., A novel and universal method for microRNA RT-gPCR data normalization, Genome Biology 10:R64 (Jun. 16, 2009).

The miRNA profile is evaluated for the presence or absence of a miRNA signature indicative of MS or response to treatment. The presence or absence of the signature may be determined by any suitable algorithm, which may involve determining the presence of threshold miRNA levels that are indicative of MS or response to treatment. In some embodiments, the threshold miRNA levels are set to include (as indicative of MS) about the top or bottom 10% (e.g., top and bottom 5% to 15%) of expression levels as determined in suitable populations of MS patients and/or healthy controls. In such embodiments, the use of increasing numbers of miRNAs from Table 1, Table 2, and/or Table 3 may increase predictive value.

Alternatively or in addition, the algorithm may involve classifying a sample between MS and non-MS groups, and/or between treatment responsive and non-responsive groups. For example, samples may be classified on the basis of threshold values as described, or based upon Mean and/or Median miRNA levels in responsive patients versus a non-responsive and/or untreated populations. Various classification schemes are known for classifying samples between two or more classes or groups, and these include, without limitation: Principal Components Analysis, Naïve Bayes, Penalized Logistic Regression, Support Vector Machines, Nearest Neighbors, Decision Trees, Logistic, Artificial Neural Networks, and Rule-based schemes. In addition, the predictions from multiple models can be combined to generate an overall prediction. For example, a “majority rules” prediction may be generated from the outputs of a Naïve Bayes model, a Support Vector Machine model, and a Nearest Neighbor model.

Thus, a classification algorithm or “class predictor” may be constructed to classify samples. The process for preparing a suitable class predictor is reviewed in R. Simon, Diagnostic and prognostic prediction using gene expression profiles in high-dimensional microarray data, British Journal of Cancer (2003) 89, 1599-1604, which review is hereby incorporated by reference in its entirety.

MS and non-MS signatures, including treatment-responsive signatures, for classifying samples may be assembled from miRNA expression data, which may be stored in a database and correlated to patient profiles. Signatures may be selected for a particular patient by, for example, age, race, gender, and/or clinical manifestations of MS. The MS signatures may represent a particular clinical course of MS, such as relapsing-remitting MS, secondary-progressive MS, progessive-relapsing MS, and primary progressive MS. Such additional demographic criteria, such as age, race, gender, MS treatment, and clinical manifestation and course of MS, may be used as factors in the algorithm.

The invention thereby provides an accurate predictor for the presence and/or absence of MS, including relapsing remitting MS, or a positive response to treatment (e.g., Beta-interferon) and in some embodiments provides a positive predictive value of at least 85%, at least 90%, or at least 94%. In various embodiments, the method according to this aspect of the invention identifies a positive response to interferon treatment with at least about 50%, 75%, 80%, 85%, 90%, 95%, 97%, 98%, 99% or greater accuracy. In this respect, the method according to this aspect may lend additional or alternative predictive value over standard clinical methods of diagnosing or monitoring MS, such as for example, absence or presence of lesions on an MRI, testing positive or negative for oligoclonal bands, or the absence or presence of other signs and symptoms of MS such as blurred vision, fatigue, and/or loss of balance.

Methods for Preparing miRNA Profiles

In another aspect, the invention provides a method for preparing a miRNA profile indicative of the presence or absence of MS, or indicative of a positive response to treatment. The method comprises preparing a miRNA profile from a biofluid sample, such as a serum or plasma sample (or fraction thereof) of a patient suspected of having MS, and profiles may be prepared pre-treatment and during treatment for MS. The miRNA profile includes the level of expression of 150 miRNAs or less, and includes at least 2 miRNAs of Table 1, Table 2, and/or Table 3. In certain embodiments, the miRNA profile comprises the level of at least 4, or at least 6, or at least 8, or at least 10, or at least 20, or at least 50, or at least 75 miRNAs of Table 1, Table 2, and/or Table 3. The miRNA profile may be prepared with the use of a custom kit or array, e.g., to allow particularly for the profiling of miRNAs associated with MS. In certain embodiments, the miRNA profile includes the level of miRNAs associated with a at least one non-MS autoimmune disorder, inflammatory disorder or infectious disease, to better discriminate disease states having overlapping symptoms, such as systemic lupus erythematosus, Sjögren's syndrome, vasculitis, sarcoidosis, Behçet's disease, Lyme disease, syphilis, progressive multifocal leukoencephalopathy, herpes zoster, lysosomal disorder, adrenoleukodystrophy, and CNS lymphoma.

In some embodiments, the profiling involves determining the expression level of 150 miRNAs or less, or in other embodiments 100 miRNAs or less, 75 miRNAs or less, 50 miRNAs or less, 25 miRNAs or less, or 10 miRNAs or less, including miRNAs from Table 1, Table 2, and/or Table 3. In some embodiments, at least 25%, or at least 50%, or at least 75% of the miRNAs of the profile are listed in Table 1, Table 2, and/or Table 3.

The miRNA profile is determined by an amplification and/or hybridization-based assay, including, for example, Real-Time PCR (e.g., TaqMan). Suitable detection formats are described in more detail below. miRNA levels may be expressed in accordance with the selected detection assay. For example, where real time PCR is conducted, miRNA levels may be expressed in terms of cycle threshold (CT) values. CT values may be normalized as described herein. Alternatively, the profile may be determined by microarray analysis, and the miRNA levels expressed by relative hybridization signal intensity, as normalized for variables such as background, sample processing, and hybridization efficiency.

The method may further comprise determining the presence of at least one control RNA to normalize expression levels across samples, e.g., with an exogenously added RNA or miRNA as described (e.g., an Arabidopsis miRNA, such as ath-miR-159a, or human miRNA not expressed in the sample undergoing analysis). Alternatively or in addition, other methods of normalizing expression levels may be employed in this aspect of the invention, such as normalizing based upon the Mean or Median level of all miRNAs on a given assay run. Methods for normalizing miRNA expression levels are described in Benes and Castoldi, Expression profiling of microRNA using real-time quantitative PCR, how to use it and what is available, Methods 50:244-249 (January 2010); A novel and universal method for microRNA RT-gPCR data normalization, Genome Biology 10:R64 (Jun. 16, 2009), which are hereby incorporated by reference in their entirety.

### Assay Formats

miRNA profiles and miRNA signatures may be prepared according to any suitable method for measuring miRNA levels. That is, the profiles and signatures may be prepared using any quantitative or semi-quantitative method for determining miRNA levels in samples. Such methods include polymerase-based assays, such as Real-Time PCR (e.g., Taqman™), hybridization-based assays, for example using microarrays, nucleic acid sequence based amplification (NASBA), flap endonuclease-based assays, as well as direct RNA capture with branched DNA (QuantiGene™) Hybrid Capture™ (Digene), or nCounter™ miRNA detection (nanostring). The assay format, in addition to determining the miRNA levels will also allow for the control of, inter alia, intrinsic signal intensity variation. Such controls may include, for example, controls for background signal intensity and/or sample processing, and/or hybridization efficiency, as well as other desirable controls for quantifying miRNA levels across samples (e.g., collectively referred to as “normalization controls”). Exemplary assay formats for determining miRNA levels, and thus for preparing miRNA profiles and obtaining data for training MS signatures are described in this section.

The invention may employ reverse transcription PCR and real-time PCR. The application of fluorescence techniques to RT-PCR combined with suitable instrumentation has led to quantitative RT-PCR methods that combine amplification, detection and quantification in a closed system. Two commonly used quantitative RT-PCR techniques are the TaqMan RT-PCR assay (ABI, Foster City, USA) and the Lightcycler assay (Roche, USA). Commercial RT-PCR products for determining miRNA levels are commercially available, and include the TaqMan Low Density miRNA Array card (Applied Biosystems).

The TaqMan detection assays offer certain advantages. First, the methodology makes possible the handling of large numbers of samples efficiently and without cross-contamination and is therefore adaptable for robotic sampling. As a result, large numbers of test samples can be processed in a very short period of time using the TaqMan assay. Another advantage of the TaqMan system is the potential for multiplexing. Since different fluorescent reporter dyes can be used to construct probes, the expression of multiple miRNAs associated with MS could be assayed in the same PCR reaction, thereby reducing the labor costs that would be incurred if each of the tests were performed individually.

Expression profiling of miRNAs using real time quantitative PCR is also described in Benes and Castoldi, Expression profiling of microRNA using real-time quantitative PCR, how to use it and what is available, Methods 50:244-249 (2010); and Chen et al., Reproducibility of quantitative RT-PCR array in miRNA expression profiling and comparison with microarray analysis, BMC Genomics 10:407 (Aug. 28, 2009), each of which is hereby incorporated by reference in its entirety. Briefly, miRNAs present in the sample are converted to cDNA using miRNA-specific primers (either stem-loop or linear miRNA specific primers having a universal 5′ sequence), or by tailing or ligating the miRNAs with a common sequence for priming (e.g., using E. coli poly(A) polymerase or T4 ligase). Amplification of the cDNA may then be quantified in real time, for example, by detecting the signal from a fluorescent reporting molecule, where the signal intensity correlates with the level of DNA at each amplification cycle. Fluorescent technologies include SYBR Green (I or II), which is a DNA-intercalating dye, and TaqMan probes. TaqMan probes have fluorescent and quenching moieties within close proximity, but with the 5′→3′ exonuclease activity of Taq polymerase during amplification, the fluorescent and quencher-containing nucleotides are hydrolyzed and no longer maintained at close proximity by the probe, thereby resulting in fluorescence. In certain embodiments, the cDNA is pre-amplified (e.g., with about 5 to about 15 PCR cycles), prior to real time detection with RT-PCR.

Alternatively, the assay format may employ the methodologies described in Direct Multiplexed Measurement of Gene Expression with Color-Coded Probe Pairs, Nature Biotechnology (Mar. 7, 2008), which describes the nCounter™ Analysis System (nanoString Technologies). This system captures and counts individual RNA transcripts by a molecular bar-coding technology, and is commercialized by Nanostring.

In other embodiments, the invention employs detection and quantification of RNA levels in real-time using nucleic acid sequence based amplification (NASBA) combined with molecular beacon detection molecules. NASBA is described for example, in Compton J., Nucleic acid sequence-based amplification, Nature 1991; 350(6313):91-2. NASBA is a singe-step isothermal RNA-specific amplification method. Generally, the method involves the following steps: RNA template is provided to a reaction mixture, where the first primer attaches to its complementary site at the 3′ end of the template; reverse transcriptase synthesizes the opposite, complementary DNA strand; RNAse H destroys the RNA template (RNAse H only destroys RNA in RNA-DNA hybrids, but not single-stranded RNA); the second primer attaches to the 3′ end of the DNA strand, and reverse transcriptase synthesizes the second strand of DNA; and T7 RNA polymerase binds double-stranded DNA and produces a complementary RNA strand which can be used again in step 1, such that the reaction is cyclic.

In yet other embodiments, the assay format is a flap endonuclease-based format, such as the Invader™ assay (Third Wave Technologies). In the case of using the invader method, an invader probe containing a sequence specific to the region 3′ to a target site, and a primary probe containing a sequence specific to the region 5′ to the target site of a template and an unrelated flap sequence, are prepared. Cleavase is then allowed to act in the presence of these probes, the target molecule, as well as a FRET probe containing a sequence complementary to the flap sequence and an auto-complementary sequence that is labeled with both a fluorescent dye and a quencher. When the primary probe hybridizes with the template, the 3′ end of the invader probe penetrates the target site, and this structure is cleaved by the Cleavase resulting in dissociation of the flap. The flap binds to the FRET probe and the fluorescent dye portion is cleaved by the Cleavase resulting in emission of fluorescence.

In yet other embodiments, the assay format employs direct RNA capture with branched DNA (QuantiGene™, Panomics) or Hybrid Capture™ (Digene).

The design of appropriate primers and probes (e.g., TaqMan probes) for reverse transcribing, amplifying, or hybridizing to a particular target miRNA, and as configured for any appropriate nucleic acid detection assay, is well known.

The use of RT-PCR and microarray approaches for determining miRNA levels is described in Chen et al., Reproducibility of quantitative RT-PCR array in miRNA expression profiling and comparison with microarray analysis, BMC Genomics 10:407 (2009), which is hereby incorporated by reference.

### Computer Systems

In another aspect, the invention is a computer system that contains a database, on a computer-readable medium, of miRNA expression values determined in an MS patient population and in a non-MS patient population. These miRNA expression values are determined in biofluid samples, such as serum or plasma or fraction thereof, or in other embodiments, whole blood cell samples, white blood cell samples (e.g., PBMC samples), urine samples, or cerebrospinal fluid samples, and for miRNAs of Table 1, Table 2, and/or Table 3. The database may include, for each miRNA, Mean and/or Median MS and Mean and/or Median Control (e.g., non-MS or healthy) expression levels, as well as various statistical measures, including measures of value dispersion (e.g., Standard Variation), fold change (e.g., between control and MS populations), and statistical significance (statistical association with MS). The database in some embodiments includes threshold expression levels that are indicative of MS for each miRNA associated with MS.

The MS patient population may include patients being treated with Beta-interferon, Glatiramer acetate, and/or Natalizumab, and such treatment and other clinical information may be included in the database such that an appropriate miRNA expression signature may be trained for use with the diagnostic methods of the invention. Generally, signatures may be trained based upon parameters to be selected and input by a user, with these parameters including one or more of age, race, gender, MS treatment, and clinical manifestation and course of MS.

In certain embodiments, the database contains Mean and/or Median miRNA expression values for at least about 5, 8, 10, 20, 40, 50, or all miRNAs of Table 1, Table 2, and/or Table 3. In some embodiments, the database may contain Mean and/or Median miRNA expression levels for more than about 100 miRNAs, or more than about 300 miRNAs, or more than about 400 miRNAs, including those of Table 1, Table 2, and/or Table 3. For RT-PCR-based assays, miRNA expression levels may be expressed in terms of CT or change in CT between MS and control groups.

The computer system of the invention may be programmed to classify (e.g., in response to user inputs) a miRNA profile as a treatment responsive or non-responsive profile, based upon the miRNA expression levels stored and/or generated from the database. For example, the computer system may be programmed to perform any of the known classification schemes for classifying gene expression profiles. Various classification schemes are known for classifying samples, and these include, without limitation: Principal Components Analysis, Naïve Bayes, Penalized Logistic Regression, Support Vector Machines, Nearest Neighbors, Decision Trees, Logistic, Artificial Neural Networks, and Rule-based schemes. The computer system may employ a classification algorithm or “class predictor” as described in R. Simon, Diagnostic and prognostic prediction using gene expression profiles in high-dimensional microarray data, British Journal of Cancer (2003) 89, 1599-1604, which is hereby incorporated by reference in its entirety.

The computer system may further comprise a display, for presenting and/or displaying a result, such as a signature assembled from the database, or the result of a comparison (or classification) between input miRNA expression values and an MS signature. Such results may further be provided in a tangible form (e.g., as a printed report).

The computer system of the invention may further comprise relational databases containing information pertaining to, for instance, the miRNAs of Table 1, Table 2, and/or Table 3. For example, the database may contain information associated with a given miRNA, such as descriptive information about the underlying biology and/or pathology of a miRNA and its potential association with disease. Methods for the configuration and construction of databases and computer-readable media to which such databases are saved are widely available, for instance, see U.S. Pat. No. 5,953,727, which is hereby incorporated by reference in its entirety.

The computer system of the invention may be linked to an outside or external database (e.g., on the world wide web) such as GenBank (ncbi.nlm.nih.gov/entrez.index.html) and Sanger website for miRNAs (mirbase.org). In certain embodiments, the external database is GenBank and the associated databases maintained by the National Center for Biotechnology Information (NCBI) (ncbi.nlm.nih.gov), including PubMed.

### Diagnostic Kits and Tests

The invention further provides a kit or test for preparing miRNA profiles as described herein. Such miRNA profiles comprise the absolute or relative level (or abundance) of miRNAs present in a sample, and include the levels for a plurality of miRNAs of Table 1, Table 2, and/or Table 3. In various embodiments, the kit is configured to determine the level of at least about 4, 6, 8, 10, 20, 50 or more miRNAs of Table 1, Table 2, and/or Table 3.

The kit may be a custom test or array, e.g., to allow particularly for the profiling of miRNAs associated with MS as described. For example, the kit may comprise probes and/or primers specific for the detection of 150 miRNAs or less, or in other embodiments 100 miRNAs or less, 75 miRNAs or less, 50 miRNAs or less, 25 miRNAs or less, including 4, 6, 8, 10, 20, 50 or more miRNAs of Table 1, Table 2, and/or Table 3.

The test or kit may be configured for a detection system described herein, including RT-PCR (e.g., TaqMan). For example, the kit or test may comprise miRNA-specific primers and/or TaqMan probes for 4, 6, 8, 10, 20, 50 or more miRNAs of Table 1, Table 2, and/or Table 3. Alternatively, the kit may comprise miRNA-specific primers for the miRNAs of Table 1A, Table 2, and/or Table 3, and SYBR Green dye (I or II) for detecting amplified miRNAs. Such kits may further include reagents or tools for miRNA isolation from samples, cDNA preparation (e.g., reverse transcriptase), and PCR amplification (e.g., Taq polymerase).

The primers and/or probes may be designed to detect gene expression levels in accordance with any assay format, including those described herein under the heading “Assay Format.” Exemplary assay formats include polymerase-based assays, such as RT-PCR, TaqMan™, hybridization-based assays, for example using DNA microarrays or other solid support, nucleic acid sequence based amplification (NASBA), flap endonuclease-based assays.

The kit or test may further comprise one or more normalization controls. For example, the normalization control may be an exogenously added RNA or miRNA that is not naturally present in the sample. The normalization control in certain embodiments is an Arabidopsis miRNA, such as ath-miR-159a, or one or more human miRNAs that are not expressed in the sample undergoing analysis (e.g., serum). In such embodiments, the test may further provide miRNA-specific primers for reverse transcribing and/or amplifying the normalization control(s), and a TaqMan probe specific therefore.

The design of miRNA-specific primers (e.g., with a Tm in the range of about 50° C. to about 65° C.) is described in Benes and Castoldi, Expression profiling of microRNA using real-time quantitative PCR, how to use it and what is available, Methods 50:244-249 (2010), which is hereby incorporated by reference in its entirety. The miRNA nucleotide sequences, for designing miRNA-specific primers, are known.

Without further description, it is believed that one of ordinary skill in the art can, using the preceding description and the following illustrative examples, make and use the present invention.

### EXAMPLES

### Serum Collection

Blood is collected in Serum Separator Tubes (BD, 8-9 ml of blood each). The tubes are inverted five times and the blood is allowed to clot for at least 30 minutes before centrifugation to separate the serum from the blood cells. Tubes are centrifuged according to the manufacturer's recommendations within 2 hours after blood collection. Serum is carefully removed from the tube and 0.5 mL aliquots are transferred to barcode-labeled plastic cryovials and frozen for storage and shipment.

Serum microRNA Profiling

Individual serum aliquots are processed using the TaqMan Low Density Array (“TLDA card”) platform (Life Technologies—Applied Biosystems) to produce miRNA expression profiles. There are two human TLDA cards, A and B, that cover a total of 667 unique human miRNAs. The A card includes TaqMan assays for 377 individual human miRNAs and 4 control miRNAs (381 total assays). The B card includes TaqMan assays for 290 individual human miRNA (381 total assays). One of the control assays is for a non-human miRNA, ath-miR-159a, which can be used as a negative control or to control for variable RNA recovery during the isolation of miRNA from individual serum samples. To simplify sample processing, pools of RT and PCR primers specific for the individual miRNA on each TLDA card are available. Separate “Megaplex” RT and PreAmp primer pools are available for the TLDA A and B cards that contain all the primers required to amplify all the targets included on each of the two cards. The Megaplex RT pools are used to convert miRNA targets to cDNA and the Megaplex PreAmp primer pools are used to amplify the DNA targets prior to TaqMan analysis. The “preamplification” step increases sensitivity of the assay and allows for the detection of miRNAs present at copy numbers too low to be detected using standard TaqMan assays.

Circulating RNA is isolated from 200 uL of serum using a modified RNA isolation protocol based on the mirVana Paris miRNA Isolation kit (Life Technologies—Ambion). A fixed concentration of synthetic ath-miR-159a oligonucleotide is added to the 2× Denaturing Solution provided in the mirVana kit and spiked into each serum sample. Alternatively, or in addition, samples may be spiked with hsa-miR-509-3-5p, hsa-miR-615-5p, or hsa-miR-875-3p, which are not detectable in human serum with this platform. RNA is converted to cDNA using Megaplex RT primer pools for the TLDA A and/or B card (Life Technologies—Applied Biosystems) and amplified prior to TagMan analysis using Megaplex PreAmp primer pools for the TLDA A and/or B card and 12-14 cycles of PCR. The resulting amplified DNA is then applied to TLDA A and/or B cards for TagMan analysis.

### A Comparison of microRNA Expression Profiles of RRMS Patients Who Are Not on Immunomodulating Treatment With Those Who Are on Treatment with IFN-Beta 1a-Example 1

MicroRNA data was generated using Applied Biosystems Human miRNA TLDA “A” cards (Part #4398965) for serum samples from 119 RRMS patients. As the assays on these cards are RT-PCR “Taqman” based, the data is represented as “threshold cycle” (Ct) values. Each sample was assigned to one of two groups, “RRMS patients not on treatment” or “RRMS patients on treatment.” One hundred of these patients were not on immunmodulating treatment for this disease at the time of the draw (either treatement naive or washed-out). Nineteen patients were on Avonex® treatment. Prior to analysis, the data was normalized as follows:

1. For each card, i.e., each sample, a mean Ct value was generated for all microRNAs on the card detected at a level of less than 35 Cts, and

2. The mean Ct value was subtracted from the Ct values for each individual miRNA on that card.

Of the 381 microRNA assays present on the cards, data for four of these were removed (ath-miR-159a, hsa-miR-509-3-5p, hsa-miR-615-5p, and hsa-miR-875-3p) as they were used as postive controls.

A Mann-Whitney U Test (Partek Genomics Suite® 6.5) was performed on the normalized microRNA data from these two groups. p-values (uncorrected and corrected for ties) and median values for each group were calculated. A step-up false discovery rate correction (a.k.a Benjamini and Hochberg false discovery rate correction) was applied to the p-values corrected for ties. Also, a “bootstrap” analysis was performed (200 trials) to generate an additional p-value. Table 1 below lists the miRNAs and Median expression levels with a step-up p-value of 0.05 or less.

TABLE 1
microRNA signature

| | | | Median | Median |
|---|---|---|---|---|
| miR | p-value | stepup(p-value) | (Avonex) | (Negative) |
| hsa-miR-222-4395387 | 1.55E−07 | 2.92E−05 | −7.81 | −6.55693 |
| hsa-miR-454-4395434 | 1.55E−07 | 2.92E−05 | −3.61 | −1.9949 |
| hsa-miR-346-4373038 | 1.11E−06 | 0.00013949 | 15 | 5.1305 |
| hsa-let-7b-4395446 | 5.31E−06 | 0.000500467 | −4.29 | −2.68347 |
| hsa-miR-574-3p-4395460 | 2.29E−05 | 0.00172666 | −0.63 | 1.50087 |
| hsa-miR-186-4395396 | 3.14E−05 | 0.00197297 | −5.75 | −4.65053 |
| hsa-let-7c-4373167 | 4.17E−05 | 0.00224584 | 1.53 | 2.70375 |
| hsa-let-7g-4395393 | 4.87E−05 | 0.00229499 | −3.15 | −1.95509 |
| hsa-miR-126-4395339 | 6.43E−05 | 0.00269346 | −8.59 | −7.99626 |
| hsa-miR-374a-4373028 | 0.000144 | 0.00540367 | −4.35 | −3.62094 |
| hsa-miR-146b-5p-4373178 | 0.000162 | 0.00540367 | −4.52 | −3.47773 |
| has-miR-155-4395459 | 0.000172 | 0.00540367 | −0.45 | 0.227386 |
| hsa-miR-323-3p-4395338 | 0.000242 | 0.00670521 | 2.91 | 2.29246 |
| hsa-miR-125a-5p-4395309 | 0.000249 | 0.00670521 | 0.58 | 1.63609 |
| hsa-let-7e-4395517 | 0.00032 | 0.00804267 | −4.66 | −3.47845 |
| hsa-miR-182-4395445 | 0.000358 | 0.00843538 | 3.66 | 5.01563 |
| hsa-miR-32-4395220 | 0.000554 | 0.0122858 | 2.76 | 4.73444 |
| hsa-miR-202-4395474 | 0.000803 | 0.0168184 | 4.22 | 15 |
| hsa-miR-628-5p-4395544 | 0.000976 | 0.0191328 | 1.2 | 2.08315 |
| hsa-miR-196b-4395326 | 0.001015 | 0.0191328 | 3.28 | 6.18173 |
| hsa-miR-142-3p-4373136 | 0.001343 | 0.02411 | −5.14 | −4.4664 |
| hsa-miR-200c-4395411 | 0.002098 | 0.0359521 | 1.66 | 2.2262 |
| hsa-miR-15a-4373123 | 0.002544 | 0.0416995 | 4.69 | 10.7644 |
| hsa-miR-653-4395403 | 0.0029 | 0.0455542 | 15 | 5.12371 |

### A Comparison of MicroRNA Profiles of Relapsing Remitting Multiple Sclerosis (RRMS) Patients Who Are Not on Immunomodulating Treatment With Those on Treatment With INF-Beta 1a-Example 2

MicroRNA data was generated (as described) using Applied Biosystems Human miRNA TLDA “A” cards (Part #4398965) for serum samples from 117 RRMS patients (including those from Example 1). As the assays on these cards are RT-PCR “Taqman”-based, the data is represented as Ct (Cycle threshold) values. Each sample was assigned to one of two groups as above. Of these patients, 78 were not on immunmodulating treatment for this disease at the time of the draw (i.e. they were either treatement naive or had been treatment negative for at least six months). The remainder of the patients (39) were on Avonex® (INF-beta 1a) treatment at the time of sample draw.

Prior to analysis, the data was normalized as follows: (1) For each card, a mean Ct value was generated for all microRNAs on the card expressed at a level of less than 35 Cts, and (2) The mean was subtracted from all Ct values for that card. Those microRNAs with a raw Ct value of greater than 35 were arbitrarily set to a value of 15 after normalization. Of the 381 microRNA assays present on the cards, data for four of these were removed (ath-miR-159a, hsa-miR-509-3-5p, hsa-615-5p, and hsa-875-3p) as they were used as postive controls. Data for another microRNA—hsa-miR-509-5p—was also removed as there was likely cross-hybridization with one of the positive controls.

A Mann-Whitney U Test (Partek Genomics Suite® 6.5) was performed on the microRNA normalized data from these two groups and p-values and median values for each group were generated. A step-up false discovery rate correction (a.k.a Benjamini and Hochberg false discovery rate correction) was applied to the p-values. The significant microRNAs—defined as those having a step-up false discovery rate corrected value of less than or equal to 0.05—along with p-values and normalized median expression are listed in Table 2.

TABLE 2
miRNA signature

| | | | Normalized | |
|---|---|---|---|---|
| | | | Median | Normalized |
| microRNA TaqMan | | | (Avonex | Median (Negative |
| Assay | p-value | Step-up(p-value) | Treated) | Treatment) |
| hsa-miR-222-4395387 | 1.89E−13 | 7.19E−11 | −7.61484 | −6.65372 |
| hsa-miR-365-4373194 | 9.69E−09 | 1.85E−06 | 1.1 | −0.0847311 |
| hsa-miR-454-4395434 | 4.28E−08 | 4.78E−06 | −3.18391 | −2.27143 |
| hsa-miR-140-5p-4373374 | 5.02E−08 | 4.78E−06 | −4.48701 | −3.86503 |
| hsa-miR-323-3p-4395338 | 1.69E−07 | 1.29E−05 | 3.3 | 2.35493 |
| hsa-miR-618-4380996 | 4.71E−07 | 2.56E−05 | 3.64719 | 15 |
| hsa-let-7b-4395446 | 5.62E−07 | 2.68E−05 | −3.99 | −2.96482 |
| hsa-miR-142-3p-4373136 | 1.69E−06 | 7.16E−05 | −5.33554 | −4.73711 |
| hsa-miR-628-5p-4395544 | 5.38E−06 | 0.000204839 | 0.87 | 1.74468 |
| hsa-miR-146b-5p-4373178 | 1.19E−05 | 0.000410737 | −4.30161 | −3.72018 |
| hsa-miR-653-4395403 | 1.92E−05 | 0.000610159 | 15 | 4.88607 |
| hsa-miR-29b-4373288 | 2.19E−05 | 0.000640586 | 3.11219 | 4.455 |
| hsa-miR-296-5p-4373066 | 8.46E−05 | 0.00225433 | 3.54 | 2.76742 |
| hsa-miR-328-4373049 | 8.88E−05 | 0.00225433 | −2.83 | −3.36766 |
| hsa-miR-200a-4378069 | 0.000109948 | 0.00261814 | 6.19836 | 15 |
| hsa-miR-346-4373038 | 0.000135864 | 0.00304495 | 6.93 | 5.32276 |
| hsa-miR-491-5p-4381053 | 0.00015265 | 0.00323109 | 0.42 | 0.991089 |
| has-miR-155-4395459 | 0.000315865 | 0.00633392 | −0.476542 | 0.0758827 |
| hsa-miR-590-5p-4395176 | 0.000448962 | 0.00855273 | −3.71 | −3.25973 |
| hsa-miR-95-4373011 | 0.000569096 | 0.00996162 | 3.26 | 2.45492 |
| hsa-miR-99a-4373008 | 0.000575212 | 0.00996162 | 2.68244 | 3.53442 |
| hsa-miR-551b-4380945 | 0.000689001 | 0.0114087 | 15 | 15 |
| hsa-miR-204-4373094 | 0.000718659 | 0.0114087 | 2.04396 | 1.37061 |
| hsa-miR-518f-4395499 | 0.000798075 | 0.0121627 | 15 | 15 |
| hsa-miR-29a-4395223 | 0.000832064 | 0.0121929 | −3.88848 | −3.42588 |
| hsa-miR-502-3p-4395194 | 0.00106586 | 0.0150405 | 4.0812 | 3.23474 |
| hsa-miR-744-4395435 | 0.00115642 | 0.015348 | −1.25741 | −1.88306 |
| hsa-miR-494-4395476 | 0.00116822 | 0.015348 | 2.4 | 1.48172 |
| hsa-miR-202-4395474 | 0.00135923 | 0.0172622 | 4.56 | 5.9696 |
| hsa-miR-93-4373302 | 0.00162634 | 0.0197504 | −5.38 | −5.00486 |
| hsa-miR-330-3p-4373047 | 0.00165883 | 0.0197504 | 4.66 | 4.12321 |
| hsa-miR-642-4380995 | 0.0017599 | 0.0203189 | 4.53902 | 3.55077 |
| hsa-miR-99b-4373007 | 0.00226674 | 0.0246751 | 1.17246 | 0.599625 |
| hsa-miR-146a-4373132 | 0.00240128 | 0.0254135 | −7.53553 | −7.1999 |
| hsa-miR-186-4395396 | 0.00290426 | 0.029906 | −5.45661 | −4.83874 |
| hsa-miR-15a-4373123 | 0.00307291 | 0.0305883 | 4.16 | 5.5907 |
| hsa-miR-126-4395339 | 0.00313108 | 0.0305883 | −8.48 | −8.118 |

Clustering analysis indicated that nine of the Avonex-treated patient samples were potential outliers. Therefore, a second set of statistics were generated using the procedures described above, except that the data for these nine patients was excluded. Table 3 lists the significant microRNAs.

TABLE 3
miRNA signature

| | | | Normalized | |
|---|---|---|---|---|
| | | | Median | Normalized |
| | | | (Avonex | Median (Negative |
| microRNA TaqMan Assay | p-value | Step-up(p-value) | Treated) | Treatment) |
| hsa-miR-222-4395387 | 9.90E−12 | 3.77E−09 | −7.67882 | −6.65372 |
| hsa-miR-454-4395434 | 1.29E−08 | 2.46E−06 | −3.32774 | −2.27143 |
| hsa-miR-140-5p-4373374 | 1.37E−06 | 0.000174178 | −4.4685 | −3.86503 |
| hsa-miR-186-4395396 | 5.18E−06 | 0.000492935 | −5.67408 | −4.83874 |
| hsa-miR-365-4373194 | 9.50E−06 | 0.000642699 | 0.796723 | −0.0847311 |
| hsa-miR-146b-5p-4373178 | 1.01E−05 | 0.000642699 | −4.34777 | −3.72018 |
| hsa-miR-142-3p-4373136 | 2.00E−05 | 0.00109042 | −5.41 | −4.73711 |
| hsa-let-7b-4395446 | 2.88E−05 | 0.00137271 | −3.8094 | −2.96482 |
| hsa-miR-126-4395339 | 3.35E−05 | 0.00141729 | −8.6385 | −8.118 |
| hsa-miR-296-5p-4373066 | 4.92E−05 | 0.00180552 | 3.55464 | 2.76742 |
| hsa-miR-323-3p-4395338 | 5.21E−05 | 0.00180552 | 2.94825 | 2.35493 |
| hsa-miR-618-4380996 | 0.00011 | 0.0034842 | 3.965 | 15 |
| hsa-miR-202-4395474 | 0.000137 | 0.00402017 | 4.275 | 5.9696 |
| has-miR-155-4395459 | 0.000162 | 0.00440496 | −0.608045 | 0.0758827 |
| hsa-miR-653-4395403 | 0.00023 | 0.00548718 | 15 | 4.88607 |
| hsa-miR-590-5p-4395176 | 0.000263 | 0.00590425 | −3.75731 | −3.25973 |
| hsa-miR-346-4373038 | 0.000317 | 0.00652981 | 7.01 | 5.32276 |
| hsa-miR-328-4373049 | 0.000326 | 0.00652981 | −2.63992 | −3.36766 |
| hsa-miR-99a-4373008 | 0.000575 | 0.0109509 | 2.2709 | 3.53442 |
| hsa-miR-32-4395220 | 0.00097 | 0.0175898 | 3.1065 | 4.3088 |
| hsa-miR-200a-4378069 | 0.001327 | 0.0229811 | 6.55009 | 15 |
| hsa-miR-450b-5p-4395318 | 0.002516 | 0.0416701 | 4.70343 | 15 |
| hsa-miR-628-5p-4395544 | 0.002753 | 0.0437065 | 1.09 | 1.74468 |

All patents or publications disclosed herein are incorporated by reference in their entireties.

1-71. (canceled)

72. A method for evaluating a patient's response to treatment with an immunomodulatory agent used to manage a demyelinating disease, comprising:
preparing a miRNA profile from a biofluid sample collected from the patient, and determining the presence or absence of a miRNA signature indicative of a patient's response to treatment with the immunomodulating agent, the miRNA profile comprising the measured level of at least 4 miRNAs of Table 1, Table 2, and/or Table 3.

73. The method of claim 72, wherein the patient has MS, and is being treated with beta-interferon.

74. The method of claim 72, wherein the miRNA profile is determined prior to treatment, and after treatment.

75. The method of claim 72, wherein the miRNA profile is determined in a serum or plasma sample.

76. The method of claim 75, wherein the sample is a serum sample collected with a serum separator tube.

77. The method of claim 72, wherein the miRNA profile comprises the level of at least 5 miRNAs of Table 1, Table 2, and/or Table 3.

78. The method of claim 77, wherein the miRNA profile comprises the level of at least 10 miRNAs of Table 1, Table 2, and/or Table 3.

79. The method of claim 77, wherein the miRNA profile comprises the level of at least 20 miRNAs of Table 1, Table 2, and/or Table 3.

80. The method of claim 77, wherein the miRNA profile comprises the level of at least 50 miRNAs of Table 1, Table 2, and/or Table 3.

81. The method of claim 72, wherein the miRNA profile comprises the level for 150 miRNAs or less.

82. The method of claim 72, wherein the miRNA profile comprises the level for 100 miRNAs or less.

83. The method of claim 72, wherein the miRNA profile comprises the level for 75 miRNAs or less.

84. The method of claim 72, further comprising, determining the level of one or more normalization controls in the sample.

85. The method of claim 84, wherein the sample is spiked with the normalization control(s).

86. The method of claim 85, wherein the normalization control is a non-endogenous RNA or miRNA, or a miRNA not detectable in the sample.

87. The method of claim 72, wherein miRNA levels are normalized to the Mean or Median detection levels for all miRNAs in the profile.

88. The method of claim 72, wherein miRNA profile is determined by amplification and/or hybridization-based assay.

89. A method for preparing a miRNA profile indicative of a patient's response to immunomodulating therapy for a demyelinating disease, comprising:
preparing a miRNA profile from a biofluid sample collected from a patient suspected of having MS, the miRNA profile comprising the measured level of 150 miRNAs or less including at least 4 miRNAs of Table 1, Table 2, and/or Table 3.

90. The method of claim 89, wherein the patient has MS and is being treated with beta-interferon.

91. The method of claim 90, wherein the miRNA profile is determined prior to treatment, and after treatment.

92. A kit for preparing a miRNA profile indicative of a responsive to immunomodulating therapy for a demyelinating disease, comprising: a miRNA-specific primer for reverse transcribing or amplifying each of 150 miRNAs or less, including at least 4 miRNAs of Table 1, Table 2, and/or Table 3.
